(19) United States
(12) Patent Application Publication
(10) Pub. No.: US 2022/0133602 A1
(43) Pub. Date: May 5, 2022

(54) SOLID COSMETIC COMPOSITION THAT GLIDES ON AND MELTS AWAY ON APPLICATION

(71) Applicant: CHANEL PARFUMS BEAUTE, NEUILLY SUR SEINE (FR)

(72) Inventor: Chantal MALVEZIN, PANTIN CEDEX (FR)

(73) Assignee: CHANEL PARFUMS BEAUTE, NEUILLY SUR SEINE (FR)

(21) Appl. No.: 17/434,125

(22) PCT Filed: Feb. 25, 2020
(86) PCT No.: PCT/FR2020/050356

§ 371 Date: Aug. 26, 2021

(30) Foreign Application Priority Data
Feb. 26, 2019 (FR) 19 01976

(51) Int. Cl. A61K 8/02; A61K 8/36; A61K 8/60; A61K 8/92; A61Q 1/06

(52) U.S. Cl. CPC A61K 8/0229; A61K 8/361; A61K 8/60; A61K 2800/591; A61Q 1/06; A61K 2800/43; A61K 2800/31; A61K 8/922

(57) ABSTRACT

A solid cosmetic composition including at least one lipophilic gelling agent chosen from sucrose polyesters, at least one fatty substance having a melting point between 25° C. and 55° C., at least one oil, at least one colorant. Also, a stick including the solid cosmetic composition, a method for preparing the solid cosmetic composition and a method for making up the skin, mucous membranes or integuments, including the application of the solid cosmetic composition.

### TECHNICAL FIELD

The invention relates to the field of cosmetic products, and more particularly solid cosmetic compositions such as lipsticks.

### PRIOR ART

The use of lipsticks as a means for colouring lips has undergone rapid growth, so that lipsticks are now widespread beauty products. A cosmetically acceptable lipstick must spread easily, be of even colour and have a melting point higher than the body temperature. Moreover, it must give the lips a smooth but non-fatty appearance and must keep its consistency without phenomena of exudation, sweating, rupture or disaggregation occurring.

### Technical Problem

A lipstick must not only have the qualities mentioned above but must especially give the lips a smooth creamy sensation and protect them from drying or chapping.

It has been observed that some lipsticks had a tendency to migrate, after application to the lips, towards the periphery of the mouth and in particular in the perilabial wrinkles. This phenomenon is particularly visible in the case of products intended to confer on the lips not only gloss but also an intense colour, and which consequently contain a high level of colorants. However, this migration phenomenon is obviously not desirable since one of the prime qualities that lip make-up products must satisfy is obtaining a sharp contour of the coloured mouth.

It is certainly known that using structuring agents of the wax and/or filler type substantially reduces these migration problems. However, these structuring agents negatively affect the gliding of the composition onto the lips and have a tendency to produce a matt effect that is not desirable for lip glosses.

It would therefore be desirable to be able to have available lip make-up products having lower migration than the known products, or even no migration effect, without negatively affecting the gliding of the composition onto the lips and without producing a matt effect.

Similarly, it would be useful to have available solid eye make-up products, such as eyeliners or eye shadow, which do not substantially migrate into the folds of the eyelids or into the eye contour wrinkles without negatively affecting the gliding of the composition onto the eyelid and without producing a matt effect.

The applicant has had the credit of managing to develop a combination of fatty-phase structuring agents making it possible to reduce or even prevent the migration of make-up products, without affecting the gloss of the make-up obtained or the gliding on when applied, while providing a melting sensation when applied. The applicant has also demonstrated that the compositions according to the invention increased the staying power of the make-up. All these properties help to guarantee a sharp precise application of the make-up composition, in particular on the lips, and to keep a sharp contour for the coloured lips throughout the day.

This combination of fatty-phase structuring agents can be in the form of a cast product, for example in a stick.

The object of the present invention is thus, according to a first aspect, a solid cosmetic composition comprising:
at least one lipophilic gelling agent selected from sucrose polyesters,
at least one fatty agent having a melting point between 25° C. and 55° C.,
at least one oil,
at least one colorant,
at least one wax, in a proportion of less than 9% by weight, preferably 7 to 8% by weight, with respect to the total weight of the composition, and
at least one lipophilic gelling agent other than sucrose polyester, in a proportion of less than 2% by weight, with respect to the total weight of the composition.

Another object of the invention, according to a second aspect, is a method for making up the skin, the mucous membranes or the integuments, comprising the application of said composition.

Lipophilic Gelling Agent

The term “lipophilic gelling agent” designates, in the context of the present application, a substance capable of solidifying or gelatinising the oil present in the composition of the invention.

The lipophilic gelling agent is selected from sucrose polyesters. Mention can be made of sucrose and fatty-acid esters, and preferably esters of sucrose, of stearic acid and of acetic acid, such as sucrose tetrastearate triacetate (according to the INCI name) available under the trade name Sisterna® A10E-C from the company Sisterna.

The lipophilic gelling agent of the sucrose polyester type is present in a proportion of between 5% and 10% by weight, with respect to the total weight of the composition.

Fatty Agent Having a Melting Point of Between 25° C. and 55° C.

The fatty agent having a melting point of between 25° C. and 55° C. is selected from hydrogenated oils solid at 25° C. or fatty esters solid at 25° C. and mixtures thereof.

Among the hydrogenated oils solid at 25° C., mention can be made of hydrogenated ricin oil, hydrogenated palm oil, hydrogenated suet and hydrogenated coconut oil.

Among the fatty esters solid at 25° C., mention can be made of propylene glycol myristate, myristyl myristate and cetyl alcohol.

The fatty agent having a melting point of between 25° C. and 55° C. is preferably hydrogenated coconut oil.

The fatty agent having a melting point of between 25° C. and 55° C. is present in a proportion of between 7% and 15% by weight, with respect to the total weight of the composition.

Oil

Within the meaning of the present invention, “oil” means a compound liquid at ambient temperature (25° C.) and which, when it is introduced at the rate of at least 1% by weight in water at 25° C., is not at all soluble in the water, or soluble to the extent of less than 10% by weight, with respect to the weight of oil introduced into the water.

Non-Volatile Oil

“Non-volatile oil” means an oil that has a boiling point generally above 300° C. at 760 mm of Hg (101325 Pa) and which does not exhibit any vapour tension, or only a little.

The non-volatile oils may in particular be selected from non-volatile silicone oils, non-volatile hydrocarbon oils and mixtures thereof.

“Silicone oil” means an oil comprising at least one silicon atom, and in particular at least one Si—O group.

As non-volatile silicone oil, mention can be made of the polydimethylsiloxanes containing at least 8 silicon atoms, the polyalkylmethylsiloxanes the alkyl chain of which contains 8 to 20 carbon atoms, and the oils identified by the INCI name phenyl trimethicone.

“Hydrocarbon oil” means an oil containing hydrogen and carbon atoms.

Mention can be made for example of hydrocarbons such as squalane, phytosqualane, polybutene, hydrogenated polyisobutene, hydrogenated polydecene, synthetic polyesters also known as “ester oils” and polyethers, in particular polyesters of C6-C20 acids and of C6-C20 alcohols, advantageously branched such as isononyl isononanoate; vegetable oils, branched and/or unsaturated fatty acids; branched and/or unsaturated fatty alcohols such as octyl dodecanol; or mixtures thereof.

“Ester oil” means a mono-, di-, tri- or tetra-ester. Ester oils are obtained by reacting a mono-, di-, tri- and more generally a polyol with a mono- di- tri- and more generally a polycarboxylic acid, said reagents being able to be linear or branched, saturated or unsaturated, aliphatic or aromatic, and being able optionally to comprise alkoxylated groups. Ester oils may in particular by hydroxylated.

In particular, the non-volatile ester oil may comprise 18 to 70 carbon atoms.

The non-volatile ester oil may in particular be selected from:
monoesters comprising 18 to 40 carbon atoms, in particular monoesters of formula R1COOR2 wherein R1 represents the remainder of a linear or branched fatty acid including 6 to 20 carbon atoms and R2 represents a hydrocarbon chain, in particular branched, containing 6 to 20 carbon atoms, such as for example Purcellin oil (cetostearyl octanoate), isononyl isononanoate, isodecyl neopentanoate, C12 to C15 alkyl benzoates, 2-ethylhexyl palmitate, octyldodecyl neopentanoate, 2-octyldodecyl stearate, 2-octyldodecyl erucate, isostearyl isostearate, 2-octyldodecyl benzoate, alkyl octanoates, decanoates or ricinoleates, isopropyl myristate, isopropyl palmitate, butylstearate, hexyl laurate, 2-ethylhexyl palmitate, 2-hexyldecyl laurate, 2-octyldecyl palmitate, 2-octyldodecyl myristate, and 2-diethylhexyl succinate;
diesters comprising 18 to 60 carbon atoms, in particular 18 to 50 carbon atoms, such as carboxylic diacid and monoalcohol diesters, such as diisostearyl malate; glycol and carboxylic monoacid diesters, such as neopentylglycol diheptanoate or polyglyceryl-2 diisostearate;
triesters comprising 35 to 70 carbon atoms, such as carboxylic triacid triesters, such as triisostearyl citrate or tridecyl trimellitate; or glycol and carboxylic monoacid triesters such as polyglyceryl-2 triisostearate;
tetraesters comprising 35 to 70 carbon atoms, such as pentaerythritol or polyglycerol tetraester and a carboxylic monoacid tetraester, for example pentaerythrityl tetrapelargonate, pentaerythrityl tetraisostearate, pentaerythrityl tetraisononanoate, glyceryl tridecyl-2 tetradecanoate, polyglyceryl-2 tetraisostearate or pentaerythrityl tetradecyl-2 tetradecanoate;
polyesters obtained by condensation of unsaturated fatty acid dimer and/or trimer and of diols such as those described in the patent application FR 0 853 634, such as dilinoleic acid and 1,4-butanediol polyester;

esters and polyesters of diol dimer and of mono- or dicarboxylic acid, such as diol dimer and fatty acid esters and dimer diol and dimer carboxylic diacid esters, in particular those obtained from a dimer of a C8 to C34 unsaturated fatty acid dimer, in particular C12 to C22, especially C16 to C20, and more particularly C18, such as esters of dilinoleic diacids and of dilinoleic diol dimers, for example those sold by the company Nippon Fine Chemical under the trade name LUSPLAN DD-DA5® et DD-DA7®;

triglycerides of fatty acids (liquid at ambient temperature), in particular of fatty acids having 7 to 40 carbon atoms, such as triglycerides of heptanoic or octanoic acids or jojoba oil; saturated triglycerides such as caprylic/capric triglyceride, glyceryl triheptanoate, glycerin trioctanoate, C18-36 acid triglycerides such as those sold under the reference DUB TGI 24 sold by Stearineries Dubois); and unsaturated triglycerides such as ricin oil, olive oil, ximenia oil or pracaxi oil;

or one of the mixtures thereof.

The non-volatile oil used in the present invention is preferably a glossy oil.

A glossy oil means an oil the refractive index of which is greater than 1.43 and preferably greater than 1.45, or even more preferably greater than 1.50.

The refractive index is measured by means of an ABBE Paralux refractometer ref 60-6400-9.

A non-volatile additional oil can also be used for providing supplementary properties to the composition of the invention.

By way of example, a diisostearyl malate can be added since it makes it possible to obtain good dispersion of the pigments.

Other additional oils can be added for improving the sensory properties of the formula.

According to one embodiment, the non-volatile oil is selected from squalane, caprylic capric triglyceride, isotridecyl isononanoate, pentaerythrityl tetraethylhexanoate, sorbitan isostearate, ethylhexyl palmitate, diisostearyl malate or one of the mixtures thereof.

According to a particular embodiment of the invention, the oil introduced into the composition according to the invention is a mixture of squalane, caprylic capric triglyceride, isotridecyl isononanoate, pentaerythrityl tetraethylhexanoate, sorbitan isostearate, ethylhexyl palmitate and diisostearyl malate.

The oil may be present in the composition according to the invention in a proportion of between 30% and 70% by weight, preferably between 45% and 65% by weight, more preferentially between 50% and 60% by weight, with respect to the weight of the composition.

Volatile Oil

According to an advantageous embodiment of the invention, the composition does not contain any volatile oil (0%) or very little (maximum 5% by weight with respect to the total weight of the composition).

“Volatile oil” means an oil able to evaporate in contact with the skin in less than one hour, at ambient temperature and atmospheric pressure.

The volatile oil is a volatile cosmetic oil, liquid at ambient temperature, in particular having a non-zero vapour pressure at ambient temperature and atmospheric pressure, in particular having a vapour pressure of between 0.13 Pa and 40,000 Pa (0.001 to 300 mm of Hg), preferably between 1.3 Pa and 13,000 Pa (0.01 to 100 mm of Hg), and more preferentially still between 1.3 Pa and 1300 Pa (0.01 to 1000 mm of Hg).

The volatile oils comprise volatile silicone oils and/or volatile hydrocarbon oils.

The volatile silicone oils optionally used in the compositions of the invention are linear or cyclic, in particular have from 2 to 7 silicon atoms, optionally alkyl or alkoxy groups having from 1 to 10 carbon atoms, and having a viscosity, at ambient temperature, of less than 5 cSt.

By way of examples of volatile silicone oil, mention can be made particularly of hexamethylcyclotrisiloxane, octamethylcyclotetrasiloxane, decamethylcyclopentasiloxane, dodecamethylcyclohexasiloxane, cyclotetradimethylsiloxane, cyclopentadimethylsiloxane, cyclohexadimethylsiloxane, hexamethyldisiloxane, octamethyltrisiloxane, hexylheptamethyltrisiloxane, octylheptamethyltrisiloxane, decamethyltetrasiloxane, dodecamethylpentasiloxane, heptamethylhexyl trisiloxane, heptamethyloctyl trisiloxane or one of the mixtures thereof.

Concerning the volatile hydrocarbon oil, mention can be made more particularly of a short-chain hydrocarbon oil, volatile linear alkanes as for example described in the document FR2933865 incorporated by reference.

By way of examples of short-chain hydrocarbon oils, mention can be made in particular of those selected from the group comprising isododecane, isodecane, isohexadecane, dodecane or one of the mixtures thereof.

By way of example of volatile linear alkanes, mention can be made of those having the following hydrocarbon chains:
C9-C17, C10-C14, such as a mixture of undecane and tridecane, sold by BASF Care Creations under the name Cetiol® Ultimate,
C15-19, such as those sold by Seppic under the name Emogreen L15,
C12-14, such as those sold by Biosynthis under the name Vegelight 1214LC,
C9-12 alkane, such as those sold by Daito under the name Makigreen D10.

Pasty Fatty Compound

The composition according to the invention may further comprise a pasty fatty compound that may advantageously be selected from:

lanolin and derivatives thereof,

silicone compounds, polymers or not,

fluorinated compounds, polymers or not,

vinyl polymers, in particular olefin homopolymers and copolymers, hydrogenated diene homopolymers and copolymers, linear or branched oligomers, homo or copolymers of alkyl methacrylates preferably having a C8-C30 alkyl group, homo and copolymer oligomers of vinyl esters having C8-C30 alkyl groups, homo and copolymer oligomers of vinyl esters having C8-C30 alkyl groups,

the liposoluble polyethers resulting from polyetherification between one or more C2-C100 diols, preferably C2-C50,

mixtures of beeswax and octyldodecanol such as the one sold under the name Zenibee Cream by the company Zenitech,

esters,

vegetable butters such as mango, karite, cocoa, cotton, avocado, etc. butter or one of the mixtures thereof.

Among the esters, the following can be used in particular:

esters of a glycerol oligomer, in particular diglycerol esters, especially adipic acid or glycerol condensates, for which some of the hydroxyl groups of the glycerols have reacted with a mixture of fatty acids such as stearic acid, capric acid, stearic acid and isostearic acid and 12-hydroxystearic acid, like those in particular sold under the trade name Softisan 649 by the company Sasol,

arachidyl propionate sold under the trade name Waxenol 801 by Alzo,
phytosterol esters such as the product with the INCI name “bis-behenyl/isostearyl/phytosteryl dimer dilinoleyl dimer dilinoleate” sold under the name Plandool-G by the company Nippon Fine Chemical Co, “Phytosteryl/behenyl/octyldodecyl/isostearyl lauroyl glutamate” sold under the name Eldew-PS308 by the company Ajinomoto,

fatty acid triglycerides and derivatives thereof, for example the mixture of stearyl heptanoate and stearyl caprylate sold under the name DUB Solide by the company Stéarinerie Dubois,

pentaerythritol esters,
non-crosslinked polyesters resulting from polycondensation between a dicarboxylic acid or a C4-C50 linear or branched carboxylic polyacid and a diol or a C2-C50 polyol,
the aliphatic esters resulting from the esterification of an aliphatic hydroxycarboxylic acid ester by an aliphatic carboxylic acid such as the cetyl lactate sold under the name Ceraphyl 28 by the company ISP (International Speciality Products),
the polyesters resulting from the esterification, by a polycarboxylic acid, of an aliphatic hydroxycarboxylic acid ester, said ester comprising at least two hydroxyl groups, such as the products Risocast DA-H®, and Risocast DA-L®, or one of the mixtures thereof.

Among the pasty fatty compounds, the phytosterol esters such as the product with the INCI name “bis-behenyl/isostearyl/phytosteryl dimer dilinoleyl dimer dilinoleate” sold under the name Plandool-G by the company Nippon Fine Chemical Co, “Phytosteryl/behenyl/octyldodecyl/isostearyl lauroyl glutamate” sold under the name Eldew-PS308 by the company Ajinomoto, or a mixture thereof, will preferably be selected.

The composition according to the invention may comprise a total content of pasty fatty compounds of between 1% and 40% by weight, preferably between 5% and 30% by weight, even more preferably between 10% and 20% by weight with respect to the total weight of the composition.

Colorant

The colorant may in particular be selected from hydrosoluble or liposoluble colorants, pigments, nacres, lacquers or mixtures thereof.

These colorants may optionally be surface treated by a hydrophobic agent such as silanes, silicones, fatty acid soaps, C₉₋₁₅ fluoroalcohol phosphates, acrylate/dimethicone copolymers, fluoroalcohol phosphate/silicone C₉₋₁₅ mixed copolymers, lecithins, carnauba wax, polyethylene, chitosan and amino acids, optionally acylated, such as lauroyl lysine, disodium stearoyl glutamate and aluminium acyl glutamate. The pigments may be mineral or organic, natural or synthetic.

Examples of mineral pigments are in particular titanium dioxide, iron, zinc or chromium oxides, manganese violets, ultramarines, so-called Prussian blue ferric ferrocyanide, as well as composite pigments and goniochromatic, pearlescent, interferential, photochromic or thermochromic pigments, without this list being limitative.

Examples of organic pigments that can be used in the invention are in particular carbon black, pigments of the D&C type, lacquers based on cochenille, barium, strontium, calcium or aluminium carmine or the diketopyrrolopyrroles (DPP) described in the documents EP-A-542669, EP-A-787730, EP-A-787731 and WO-A-96/08537.

The nacres may be selected from those conventionally present in make-up products, such as micas/titanium dioxide. In a variant, they may be nacres based on mica/silica/titanium dioxide, based on synthetic fluorophlogopite/titanium dioxide (SUNSHINE® from Maprecos), calcium sodium borosilicate/titanium dioxide (REFLECKS® from Engelhard) or calcium aluminium borosilicate/silica/titanium dioxide (RONASTAR® from Merck).

Advantageously, when it contains one or more pigments, the composition according to the invention furthermore contains at least one dispersing agent such as diisostearyl malate.

The colorants are present in the composition in a proportion of between 0.1% and 15%, the percentages being percentages by weight with respect to the total weight of the composition.

Wax

The term “wax” designates a fatty agent with reversible liquid/solid change, having a melting point above 55° C. and generally below 110° C., which is liquid under the conditions of preparation of the composition and which has an anisotropic crystalline organisation in the solid state.

According to one embodiment of the invention, the structuring agent is a mixture of at least one wax and at least one lipophilic gelling agent.

According to another embodiment of the invention, the structuring agent is a mixture of at least one wax and at least one silicone resin.

According to another embodiment of the invention, the structuring agent is a mixture of at least two waxes.

The appropriate wax for the cosmetic compositions of the invention comprises at least one polar wax and/or at least one apolar wax.

Polar wax means a wax comprising at least one heteroatom such as oxygen, nitrogen, silicon or phosphorus.

In particular, the polar wax may be selected from the group comprising beeswax, carnauba wax, candelilla wax, cotton wax, rice bran wax, berry wax, Chinese insect wax, montan wax, lanolin and the alcohol, acetylated, esterified and polyethoxylated derivatives thereof, kapok wax, sugarcane wax, hexyl laurate, jojoba wax, shellac wax, polyethoxylated cholesterol ether, the synthetic beeswaxes sold by Koster Keunen under the trade name Kester Wax K82H, or one of the mixtures thereof.

Mention can also be made of the vegetable ester waxes selected from the group comprising the mixture of jojoba esters, polyglycerin-3, Acacia decurrens flower wax and sunflower seed wax, said mixture being sold by Gattefosse under the trade name Acticire®, the jojoba esters sold by Floratech under the trade name Floraesters 60 or Floraesters 70, and the alkyl esters or hydrogenated alkyl esters sold by Sophim under the trade name Phytowax, such as for example the hydrogenated lauroyl oleate esters sold under the name Phytowax Olive 12L44.

Apolar wax means a hydrocarbon wax and/or a silicone wax.

“Hydrocarbon apolar wax” means a wax comprising solely carbon and hydrogen atoms and not comprising any heteroatoms such as oxygen, nitrogen, silicon or phosphorus.

Examples of hydrocarbon apolar waxes that are suitable in the compositions of the invention comprise the polyethylene wax sold by New Phase Technologies under the name Performalene 400 (P400) or by Jeen International Corporation under the name Jeenate 3H, a mixture of high molecular weight linear polyethylene and ethylene/propylene copolymer sold by Safic-Alcan under the name Lipwax® PZ80-20, a synthetic wax sold by Sasol under the name Sasol Wax C80, synthetic waxes and vegetable waxes, such as for example a mixture of synthetic wax and carnauba wax (Copernica cerifera) sold by Strahl & Pitsch under the name Smart wax 202, a mixture of synthetic wax, of Candelilla wax and of carnauba wax (Copernica cerifera) sold by Strahl & Pitsch under the name Smartwax 7743S, the Fischer Tropsch waxes sold by Cirebelle under the name Cirebelle 303, or one of the mixtures thereof.

“Silicone apolar wax” means a wax comprising a silicon heteroatom.

One example of silicone apolar wax that is suitable in the compositions of the invention is the C24-28 alkyl dimethicone sold by Evonik Industries AG under the name Abil Wax.

More particularly, according to yet another embodiment of the invention, the wax is selected from the group comprising a mixture of high molecular weight linear polyethylene and ethylene/propylene copolymer, sold by Safic-Alcan under the name Lipwax® PZ80-20, the vegetable ester waxes selected from the group comprising the mixture of jojoba esters, polyglycerin-3, Acacia decurrens flower wax and sunflower seed wax, said mixture being sold by Gattefosse under the trade name Acticire®, or one of the mixtures thereof.

The wax is present in the cosmetic composition of the invention in a proportion of less than 9% by weight, preferably from 7% to 8% by weight, with respect to the total weight of the composition.

Other Structuring Agents

The composition comprises less than 2% by weight, and is preferably free from, lipophilic gelling agent other than the saccharose polyester.

Among the lipophilic gelling agents other than the saccharose polyester used, mention can be made in particular of silicone resins, or polymeric or molecular organic or mineral lipophilic gelling agents.

By way of example of silicone resins, mention can be made of:
siloxysilicates, which may be trimethylsiloxysilicates of formula [(CH3)3SiO]x(SiO4/2)y (MQ units) wherein x and y are integers between 50 and 80,
polysilesquioxanes of formula (CH3SiO3/2).x (T units) wherein x is greater than 100 and wherein at least one of the methyl radicals may be substituted by an R group as defined above,
   the polymethylsilsesquioxanes that are polysilsesquioxanes wherein none of the methyl radicals is substituted by another group. Such polymethylsilsesquioxanes are described in the document U.S. Pat. No. 5,246,694.

By way of examples of polymethylsilsesquioxane resins commercially available, mention can be made of those that are sold:

by the company Wacker under the reference Resin MK such as Belsil PMS MK: polymer comprising CH3SiO3/2 repeating units (T units), which may also comprise up to 1% by weight (CH3)2SiO2/2 units (D units) and having a mean molecular weight of approximately 10,000,

by the company SHIN-ETSU under the references KR-220L that are composed of T units of formula CH3SiO3/2 and have Si—OH terminal groups (silanol), under the reference KR-242A. which comprise 98% T units and 2% dimethyl D units and have Si—OH terminal groups, or under the reference KR-251 comprising 88% T units and 12% dimethyl D units and have Si—OH terminal groups.

As siloxysilicate resins, mention can be made of trimethylsiloxysilicate (TMS) resins optionally in the form of powders. Such resins are sold under the reference SR1000 by the company General Electric or under the reference TMS 803 by the company Wacker. Mention can also be made of the trimethylsiloxysilicate resins sold in a solvent such as cyclomethicone, sold under the name “KF-7312J” by the company Shin-Etsu, “DC 749”, “DC 593” by the company Dow Corning.

The lipophilic gelling agents other than saccharose polyester that can be used in the compositions according to the invention may be organic or mineral, polymeric or molecular, lipophilic gelling agents.

As mineral lipophilic gelling agent, mention can be made of pyrogenated silica, optionally hydrophobic treated on the surface, the particle size of which is less than 1 μm. It is in fact possible to chemically modify the surface of the silica, by chemical reaction causing a reduction in the number of silanol groups present on the surface of the silica. It is in particular possible to substitute silanol groups by hydrophobic groups: then a hydrophobic silica is obtained. The hydrophobic groups may be:

trimethylsiloxyl groups, which are in particular obtained by treating pyrogenated silica in the presence of hexamethyldisilazane. Silicas thus treated are called “silica silylate” according to the CTFA (8^{th} edition, 2000). They are for example sold under the references Aerosil R812® by the company Degussa, and CAB-O-SIL TS-530® by the company Cabot,

dimethylsilyloxyl or polydimethylsiloxane groups, which are in particular obtained by treating pyrogenated silica in the presence of polydimethylsiloxane or dimethyldichlorosilane. Silicas thus treated are called “silica dimethyl silylate” according to the CTFA (8^{th} edition, 2000). They are for example sold under the references Aerosil R972®, and Aerosil R974® by the company Degussa, and CAB-O-SIL TS-610® and CAB-O-SIL TS-720® by the company Cabot.

Hydrophobic pyrogenated silica has in particular a particle size that may be nanometric to micrometric, for example ranging from approximately 5 to 200 nm.

The polymeric organic lipophilic gelling agents are for example the partially or completely crosslinked elastomeric organopolysiloxanes, with a three-dimensional structure, such as those sold under the names KSG6®, KSG16® and KSG18® by the company Shin-Etsu, Trefil E-505C® and Trefil E-506C® by the company Dow-Corning, Gransil SR-CYC®, SR DMF10®, SR-DC556®, SR 5CYC Gel®, SR DMF 10 Gel® and SR DC 556 Gel® by the company Grant Industries, SF 1204® and JK 113® by the company General Electric; ethylcellulose such as the one sold under the name Ethocel® by the company Dow Chemical; galactomannans including one to six, and in particular two to four, hydroxyl groups per ose, substituted by an alkyl chain, saturated or not, such as guar gum alkylated by C1 to C6 alkyl chains, and in particular C1 to C3, or one of the mixtures thereof. The sequenced copolymers of the “dibloc”, “tribloc” or “radial” type of the polystyrene/polyisoprene or polystyrene/polybutadiene type such as those sold under the name Luvitol HSB® by the company BASF, of the polystyrene/copoly(ethylene-propylene) type such as those sold under the name Kraton® by the company Shell Chemical Co or of the polystyrene/copoly(ethylene-butylene), type, the mixtures of tribloc and radial (star) copolymers in isododecane such as those sold by the company Penreco under the name VersaGel® such as for example the mixture of butylene/ethylene/styrene tribloc copolymer or ethylene/propylene/styrene star copolymer in isododecane (Versagel M 5960) or in hydrogenated polyisobutene (Versagel ME 2000).

Another type of polymeric organic lipophilic gelling agent other than sucrose polyester consists of polyamide resins or poly(ester-amide) resins, such as ester-terminated polyamides (ETPA), ester-terminated poly(ester-amides) (ETPEA), tertiary amide terminated polyamides (ATPA), polyalkyleneoxy termination polyamides (PAOPA) or polyether polyamides (PEPA).

Examples of ester-terminated polyamides (ETPA) are those identified by the INCI name “ethylenediamine/stearyl dimer dilinoleate copolymer” and available for example under the trade name Uniclear® 100VG from the company Arizona Chemical.

Examples of ester-terminated poly(ester-amides) (ETPEA) are those identified by the INCI name polyamide-8, which are “copolymers of ethylenediamine bis-stearyl ethylenediamine dimer dibenzoate/neopentylglycol/stearyl” and available for example under the trade name Oloecraft® LP-20-PA-MV from the company Croda.

Examples of tertiary amide terminated polyamides (ATPA) are those identified by the INCI name “ethylenediamine/copolymer of bis-di-C14-18 alkyl amide hydrogenated dimer diilinoate and available for example under the trade name Sylvaclear® A200V or Sylvaclear® A2614V from the company Arizona Chemical or those identified by the INCI name “diisostearyl malate and bis-dioctadecylamide dimer dilinoleic acid/ethylenediamine” and available for example under the trade name Haimalate PAM from the company Kokyu Alcohol Kogyo.

Examples of polyalkyleneoxy-terminated polyamides (PAOPA) are those identified by the INCI name polyamide-3 and available for example under the name Sylvaclear® AF1900V, Sylvaclear® PE1800V and Sylvaclear® PA1200V from the company Arizona Chemical.

Examples of polyether polyamides (PEPA) are those identified by the INCI name polyamide-6 and available for example under the name Sylvaclear® PE400V from the company Arizona Chemical.

Another type of polymeric organic lipophilic gelling agent other than sucrose polyester consists of N-acyl glutamic acid diamides. Mention can be made in particular of an N-acyl glutamic acid diamide having a straight-chain alkyl group such as dibutyl lauroyl glutamide and an N-acyl glutamic acid diamide having a branched-chain alkyl group, such as dibutyl ethylhexanoyl glutamide. Dibutyl lauroyl glutamide is available commercially as GP-1 and dibutyl ethylhexanoyl glutamide is available commercially under the name EB-21, and are both sold by Ajinomoto.

Another type of polymeric organic lipophilic gelling agent other than sucrose polyester consists of dextrin esters. Mention can be made of dextrin and fatty-acid esters, such as dextrin palmitate.

Another type of polymeric organic lipophilic gelling agent other than sucrose polyester consists of glyceryl esters. Mention can be made of eicosadioic acid diester and glycerol esterified by behenic acid. It is in particular available under the trade name NOMCORT® HK-G from the company Nisshin Oillio.

Supplementary Fillers

The composition preferably comprises less than 2% by weight with respect to the total weight of the composition, and is preferably free from, supplementary fillers.

These fillers are preferably colourless or white.

The particles that constitute it may be porous or not, and be in various forms, in particular in platelet, spherical or oblong form, whatever the crystallographic form (for example leaf, cubic, hexagonal, orthorhombic, etc.

In particular, the supplementary filler may be selected from lauroyl lysine, boron nitride, the silicone microbeads such as those sold under the name Tospearl by Toshiba for example, precipitated calcium carbonate, hydroxyapatite, particles of polyorganosiloxane elastomers, glass or ceramic microcapsules, zinc laurate, magnesium myristate, magnesium and aluminium silicate such as the one sold under the trade name Neusilin ULF2 by the company Fuji Chemical Industry, starch, a clay or one of the mixtures thereof.

Among the supplementary fillers, mention can be made of starch, a clay or one of the mixtures thereof.

The starch may be selected for example from a rice, tapioca, potato or maize starch. Rice starch is preferred, in particular the one with the INCI name distarch phosphate sold under the name “Rice PO4 Natural” by the company Agrana Starch. It absorbs oils very little and has a matt appearance and may give softness to the composition of the invention, which avoids the sought-after crunch of the particular filler as a matting agent.

The clay may be natural or synthetic. It is made lipophilic by a treatment with an alkyl ammonium salt such as a C10 to C22 ammonium chloride, for example di-stearyl di-methyl ammonium chloride. It may be selected from bentonites, in particular hectorites and montmorillonites, beidellites, saponites, nontronites, sepiolites, biotites, attapulgites and vermiculites. The clay is preferably selected from hectorites. The product sold by the name Bentone 38V CG by the company Elementis Specialties (INCI name disteardimonium hectorite) can be mentioned by way of example.

Additional Additives

Apart from the aforementioned constituents, the composition according to the invention may contain various ingredients, such as a UV filter, a vegetable or synthetic butter, a sweetener, an antioxidant, a sequestering agent, a pH adjuster, a preservative, perfumes, vitamins, hydrating agents, or one of the mixtures thereof.

The UV filters may in particular be selected from organic and inorganic filters or one of the mixtures thereof. As organic filters, mention can be made in particular of dibenzoylmethane derivatives (including butyl methoxydibenzoylmethane), derivatives of cinnamic acid (including ethylhexyl methoxycinnamate), salicylates, para-aminobenzoïc acids, β,β′-diphenylacrylates, benzophenones, benzylidene camphor derivatives, phenylbenzimidazoles, triazines, phenylbenzotriazoles and anthranilic derivatives. As inorganic filters, mention can be made in particular of filters based on mineral oxides in the form of pigments or nanopigments, coated or not, and in particular based on titanium dioxide or zinc oxide.

The composition of the invention may also contain vegetable butters or butters of synthetic origin. In all cases these are selected to avoid providing gloss. It is preferred to use butters of a matte appearance such as mango butter for example.

The composition according to the invention may also contain one or more sweeteners such as sorbitol, sucrose, xylitol, acesulfame K and sodium saccharinate; antioxidants such as alkylated or phosphorylated esters of ascorbic acid, or tocopherol and esters thereof; sequestering agents such as EDTA salts, pH adjusters; preservatives; perfumes, vitamins; hydrating agents; or one of the mixtures thereof

Examples of such adjuvants are cited in particular in the CTFA Dictionary (The International Cosmetic Ingredient Dictionary and Handbook published by The Cosmetic, Toiletry and Fragrance Association, 11th edition, 2006).

Anhydrous Composition

The composition of the invention is preferably anhydrous. “Anhydrous” means in particular that water is preferably not added deliberately to the compositions but may be present in the trace state in the various compounds used in the compositions.

Advantageously, the composition comprises less than 2% by weight water, preferably less than 0.5% by weight water, with respect to the total weight of the composition, and even more preferably is free from water.

Cast Composition

The composition of the invention is preferably cast, and in particular is in the form of a stick.

Hardness of the Composition

One of the advantages of the composition of the invention is that the texture thereof is homogeneous and creamy and that it remains so over time.

This property of stability of the texture over time is measured by means of a hardness measurement.

The cosmetic composition of the invention as defined above can also be characterised in that it has a hardness of between 200 g (grams) and 500 g (grams) and this one day after manufacture and after storage of 1 day, 15 days, 1 month and 2 months in an oven at 45° C.

The hardness of the composition, which is expressed in grams (g), is determined by measuring the compressive force measured at 20° C. by means of a texture meter sold under the name “TA-XT Plus Microstable System” by the company Swantech. The texture meter is equipped with a stainless-steel cylinder 2 mm in diameter moving at the measurement speed of 1 mm/s and penetrating the composition to a depth of 3 mm.

The hardness is the compressive force measured divided by the surface area of the cylinder of the texture meter in contact with the composition. The samples are cast hot and to the brim of a round Petri dish size 60 mm radius and 15 mm high. The samples thus prepared are stored for 24 h to 48 h at 20° C. before making the measurement.

Another object of the present invention is a solid cosmetic composition as described previously, preferably in the form of a stick, characterised in that it comprises:

5 to 10% by weight, with respect to the total weight of the composition, lipophilic gelling agent selected from sucrose polyesters, and preferably sucrose tetrastearate triacetate,

7 to 15% by weight, with respect to the total weight of the composition, a fatty agent having a melting point of between 25° C. and 55° C., and preferably hydrogenated coconut oil,

30 to 70% by weight oil, with respect to the total weight of the composition,

0.1 to 15% colorant by weight with respect to the total weight of the composition, and

optionally 5% to 40% pasty fatty compound by weight with respect to the total weight of the composition.

The lipstick composition advantageously has a glossy appearance, a homogeneous and creamy texture, melting away on application and having a particularly intense colour. These properties of the lipstick composition are revealed by sensory test.

Method for Preparing the Composition

Another object of the present invention is to provide a method for preparing a composition. This other aspect of the invention is characterised by the fact that a composition is prepared by mixing at least one lipophilic gelling agent selected from sucrose polyesters, a fatty agent having a melting point between 25° C. and 55° C., at least one oil, at least one colorant, and optionally at least one wax in a proportion of less than 9% by weight, with respect to the total weight of the composition. A composition obtained according to the method of the invention advantageously has a glossy appearance, a homogeneous and creamy texture, melting away on application and having a particularly intense colour.

Another object of the present invention is a method for preparing a composition as described previously, comprising:

(1) Grinding the pigments, if present, in a portion of the oil;

(2) Melting the wax if present, the fatty agent having a melting point between 25° C. and 55° C., the lipophilic gelling agent selected from sucrose polyesters, the remainder of the oil and any pasty fatty compound (e);

(3) Adding to the molten mixture obtained at (2) any colorants other than the pigments as well as any pigments of step (1) under continuous stirring;

(4) Optionally adding the active agents and the perfumes; and

(5) Casting the composition obtained at the step (4) hot into moulds, and then leaving to cool until solidification.

The following examples are given in order to illustrate the invention. These examples being presented only by way of illustration, the invention can in no case be limited to the object thereof.

### BRIEF DESCRIPTION OF THE DRAWINGS

FIG. 1 illustrates the result of the clinical evaluation, implemented by 3 trained evaluators on standardised photographs of 22 Caucasian women, with regard to the properties of staying power, homogeneity, intensity of the colour, intensity of gloss and migration of the composition of example 1 according to the invention.

### EXAMPLE

Lipstick compositions were prepared with the following formulae (% by weight):

TABLE 1

| | | | Invention | Comparative |
|---|---|---|---|---|
| | | | Ex 1 | Ex 2 |
| Function | Ingredient | TRADE NAME | (%) | (%) |
| Lipophilic | Sucrose | SISTERNA | 7 | 0 |
| gelling agent | tetrastearate | A10E-C | | |
| | triacetate | | | |
| Fatty agent with | Hydrogenated | HYDRABASE 32- | 9 | 0 |
| melting point | coconut oil | 34 | | |
| [25° C.-55° C.] | | | | |
| Oil | Phytosteryl/octyldod | Eldew PS203 | 11 | 15 |
| | ecyl Lauroyl | | | |
| | glutamate | | | |
| Oil | Diisostearyl malate | SALACOS 222 | 13.6 | 9.6 |
| Oil | Squalane | PHYTOSQUALAN | 13.5 | 9 |
| Oil | Caprylic capric | MIGLYOL 812 N | 7.25 | 10 |
| | triglyceride | | | |
| Oil | Isotridecyl | DERMOL 139 | 5.8 | 8 |
| | isononanoate | | | |
| Oil | Pentaerythrityl | SALACOS 5408 | 4 | 10.8 |
| | tetraethylhexanoate | | | |
| Oil | Sorbitan isostearate | SPAN 120-LQ | 1.5 | 2 |
| Wax | Copolymer of | LIPWAX PZ80-20 | 7.025 | 9 |
| | ethylene/propylene | | | |
| | and synthetic wax | | | |
| Wax | Jojoba esters & | ACTICIRE | 0.12 | |
| | polyglycerin-3 & | | | |
| | Acacia decurrens | | | |
| | flower wax & | | | |
| | Helianthus annuus | | | |
| | cera seed | | | |
| | (sunflower)seed wax | | | |
| pasty fatty | Bis-behenyl/ | PLANDOOL-G | 13 | 18 |
| substance | isostearyl/ | | | |
| | phytosteryl dimer | | | |
| | dilinoleyl dimer | | | |
| | dilinoleate | | | |
| Emollient | Meadowfoam Delta- | | 1.45 | 2 |
| | Lactone | | | |
| Antimicrobial | Lauryl PCA | | 0.725 | 1 |
| hydrator | | | | |
| Colorant | Nacres | | 0.5 | 3.35 |
| Colorant | Pigments | | 4.33 | 2.05 |
| Perfume | Perfume | | 0.08 | 0.1 |
| Active agent | | | 0.12 | 0.1 |

Preparation methodology of example 1 of the invention

(1) Grinding the pigments, in advance in a portion of the oil: diisostearyl malate;

(2) Melting the wax, the fatty agent having a melting point of between 25° C. and 55° C., the lipophilic gelling agent, the remainder of the oil and the pasty fatty compound (e);

(3) Adding, to the molten mixture obtained at (2), the nacres and the pigments of step (1) under continuous stirring;

(4) Adding the active agents or the perfumes; and

(5) Casting the composition obtained at step (4) hot in moulds, and then leaving to cool until solidification.

Preparation methodology of comparative example 2 outside the invention

(1) Grinding the pigments, in advance in a portion of the oil: diisostearyl malate;

(2) Melting the wax, the remainder of the oil and the pasty fatty compound (3);

(3) Adding, to the molten mixture obtained at (2), the nacres and the pigments of step (1) under continuous stirring;

(4) Adding the active agents or the perfumes; and

(5) Casting the composition obtained at step (4) hot in moulds, and then leaving to cool until solidification.

The composition of example 1 and the comparative composition of example 2 were evaluated from the sensory point of view by a panel of 19 trained evaluators, who allocated a mark out of 10 on the following criteria. The results are presented in table 2:

TABLE 2

| | Example 1 (according | Example 2 |
|---|---|---|
| | to the invention) | (comparative) |
| Slipperiness | 8.26 | 7.8 |
| Melting away on | 8.51 | 7.94 |
| application | | |
| Thickness of deposition | 4.57 | 3.51 |
| Opacity of deposit | 5.27 | 3.34 |
| Intensity of colour (up | 6.36 | 3.77 |
| to 5 minutes after | | |
| application) | | |

The result of these tests is that the composition according to the invention has better slipperiness and confers a melting sensation on application that is superior compared with the comparative composition, without degrading the gloss. The deposit obtained is thicker, much more opaque, and the colour is more intense up to 5 minutes after application.

The properties of staying power, homogeneity, colour intensity, gloss intensity and migration of the composition of example 1 according to the invention were moreover tested. The clinical evaluation was made by 3 trained evaluators on standardised photographs of 22 Caucasian women.

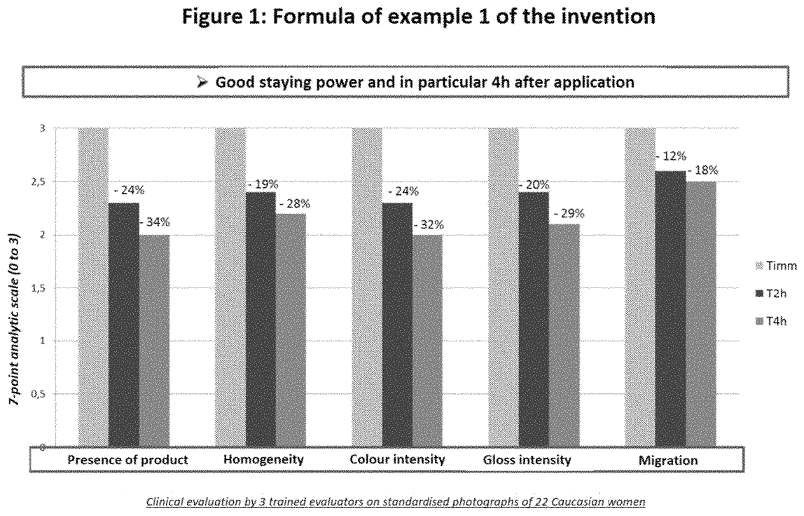

The results are presented in FIG. 1.

The composition according to the invention has very good properties of staying power, homogeneity, colour intensity, gloss intensity and migration, in particular up to 4 hours after application.

1-14. (canceled)

15. A solid cosmetic composition comprising:
at least one lipophilic gelling agent selected from sucrose polyesters,
at least one fatty agent having a melting point between 25° C. and 55° C., at least one oil,
at least one colorant,
at least one wax, in a proportion of less than 9% by weight, preferably 7 to 8% by weight, with respect to the total weight of the composition, and
at least one lipophilic gelling agent other than the sucrose polyester, in a proportion of less than 2% by weight, with respect to the total weight of the composition.

16. The composition according to claim 15, wherein the sucrose polyester is an ester of sucrose, of stearic acid and of acetic acid, and preferably the lipophilic gelling agent is sucrose tetrastearate triacetate.

17. The composition according to claim 15, wherein the lipophilic gelling agent is present in a proportion of between 5 and 10% by weight with respect to the total weight of the composition.

18. The composition according to claim 15, wherein the fatty agent having a melting point between 25 and 55° C. is selected from hydrogenated ricin oil, hydrogenated palm oil, hydrogenated suet, hydrogenated coconut oil, propyleneglycol myristate, myristyl myristate, cetyl alcohol and a mixture thereof, and preferably the fatty agent having a melting point between 25° C. and 55° C. is hydrogenated coconut oil.

19. The composition according to claim 15, wherein the fatty agent having a melting point between 25 and 55° C. is present in a proportion of between 7 and 15% by weight with respect to the total weight of the composition.

20. The composition according to claim 15, wherein the composition comprises less than 5% by weight volatile oil, and preferably the composition is free from volatile oil.

21. The composition according to claim 15, wherein the oil is present in a proportion of between 30% and 70% by weight, preferably between 45% and 65% by weight, more preferentially between 50% and 60% by weight, with respect to the total weight of the composition.

22. The composition according to claim 15, wherein the composition comprises a pasty fatty compound.

23. The composition according to claim 21, wherein the pasty fatty compound is present in a proportion of between 1% by weight and 40% by weight, preferably between 5% by weight and 30% by weight, even more preferably between 10% by weight and 20% by weight with respect to the total weight of the composition.

24. The composition according to claim 15, wherein the total colorant content is between 0.1% and 15% by weight, with respect to the total weight of the composition.

25. The composition according to claim 15, wherein the composition is free from lipophilic gelling agent other than sucrose polyester.

26. The composition according to claim 15, wherein the composition is anhydrous.

27. The composition according to claim 15, wherein the composition comprises:
5 to 10% by weight, with respect to the total weight of the composition, lipophilic gelling agent selected from sucrose polyesters, and preferably sucrose tetrastearate triacetate,
7 to 15% by weight, with respect to the total weight of the composition, a fatty agent having a melting point between 25° C. and 55° C., and preferably hydrogenated coconut oil,
30 to 70% by weight oil, with respect to the total weight of the composition,
0.1 to 15% by weight colorant, with respect to the total weight of the composition, and
optionally 5% to 40% by weight a pasty fatty compound, with respect to the total weight of the composition.

28. A method for making up the skin, mucous membranes or integuments, comprising applying the composition according to claim 15.
